(19) United States
(12) Patent Application Publication
(10) Pub. No.: US 2020/0000751 A1
(43) Pub. Date: Jan. 2, 2020

(54) CARBOXYLIC ACIDS FOR TREATING/PREVENTING NASAL CONGESTION

(71) Applicant: PROPONENT BIOTECH GMBH, Zug (CH)

(72) Inventor: Bettina ERNST, Zürich (CH)

(73) Assignee: Proponent Biotech GmbH, Zug (CH)

(21) Appl. No.: 16/097,784

(22) PCT Filed: May 17, 2017
(86) PCT No.: PCT/EP2017/061832

§ 371 Date: Oct. 30, 2018

(30) Foreign Application Priority Data
May 17, 2016 (EP) 16169882.4

(51) Int. Cl. A61K 31/19; A61K 9/00; A61K 9/08; A61P 11/02; A61P 11/04

(52) U.S. Cl. CPC A61K 31/19; A61K 9/0056; A61K 9/08; A61P 11/04; A61K 9/006; A61P 11/02; A61K 9/0043

(57) ABSTRACT

The present invention relates to a pharmaceutical composition comprising a carboxylic acid or a pharmaceutically acceptable salt thereof as active ingredient for use in treating and/or alleviating and/or preventing nasal congestion, a viral infectious disease of the respiratory tract or an inflammation of the throat. Furthermore, the present invention relates to a method for treating and/or alleviating and/or preventing nasal congestion, viral infections of the respiratory tract and/or inflammation of the throat in. a patient, comprising administering an effective amount of a carboxylic acid or a pharmaceutically acceptable salt thereof or a pharmaceutical composition comprising a carboxylic acid or a pharmaceutically acceptable salt thereof to a patient in need thereof. In addition, the present invention relates to a method for alleviating the symptoms associated with nasal congestion, viral infections of the respiratory tract and/or inflammation of the throat comprising administering an effective amount of a carboxylic acid or a pharmaceutically acceptable salt thereof or a pharmaceutical composition comprising a carboxylic acid or a pharmaceutically acceptable salt thereof to a patient in need thereof.

The present invention relates to a pharmaceutical composition comprising a carboxylic acid or a pharmaceutically acceptable salt thereof as active ingredient for use in treating and/or alleviating and/or preventing nasal congestion, chronic rhinosinusitis, a viral infectious disease of the respiratory tract or an inflammation of the throat. Furthermore, the present invention relates to a method for treating and/or alleviating and/or preventing nasal congestion, chronic rhinosinusitis, viral infections of the respiratory tract and/or inflammation of the throat in. a patient, comprising administering an effective amount of a carboxylic acid or a pharmaceutically acceptable salt thereof or a pharmaceutical composition comprising a carboxylic acid or a pharmaceutically acceptable salt thereof to a patient in need thereof. In addition, the present invention relates to a method for alleviating the symptoms associated with nasal congestion, chronic rhinosinusitis, viral infections of the respiratory tract and/or inflammation of the throat comprising administering an effective amount of a carboxylic acid or a pharmaceutically acceptable salt thereof or a pharmaceutical composition comprising a carboxylic acid or a pharmaceutically acceptable salt thereof to a patient in need thereof.

Respiratory disease is a common and significant cause of illness and death around the world. In the US, approximately 1 billion “common colds” occur each year. A study found that in 2010, there were approximately 6.8 million emergency department visits for respiratory disorders in the U.S. for patients under the age of 18; see Wier (2010) HCUP Statistical Brief #157. Agency for Healthcare Research and Quality. Rockville, Md. In 2012, respiratory conditions were the most frequent reasons for hospital stays among children; see Witt (2014) HCUP Statistical Brief #186. Rockville, Md.: Agency for Healthcare Research and Quality. In general, respiratory diseases encompass pathological conditions affecting the organs and tissues that make gas exchange possible in mammals. The respiratory tract can be divided into upper and lower respiratory tract. The upper respiratory tract, can refer to the parts of the respiratory system lying above the sternal angle (outside of the thorax), above the glottis (vocal cords), or above the cricoid cartilage. The tract consists of the nasal cavity and paranasal sinuses, the pharynx (nasopharynx, oropharynx and laryngopharynx) and sometimes includes the larynx. Due to the gas exchange with the environment in the upper respiratory tract, exposure to potentially toxic or pathogenic agents present in the environment is increased compared to other parts of the body. Therefore, many infections and diseases manifest themselves in the upper respiratory tract, as evidenced by the above numbers. There are various pharmaceuticals on the market that target diseases of the upper respiratory tract, nasal congestions and/or inflammation of the throat. Many of these pharmaceuticals cause addiction and/or habituation. Habituation may cause a decline of desired effects such as a decongestion of the nose. For this reason alone there is a constant need for alternative or improved means for treating/preventing nasal congestion and/or treating/preventing diseases of the upper respiratory tract such as viral infectious diseases and/or an inflammation of the throat.

Thus, the technical problem underlying the present invention is the provision of means and methods to treat/alleviate/prevent nasal congestion, chronic rhinosinusitis, a viral infectious disease of the respiratory tract and/or an inflammation of the throat.

The technical problem is solved by provision of the embodiments characterized in the claims.

Accordingly, the present invention relates to a carboxylic acid or a pharmaceutically acceptable salt thereof, particularly to a pharmaceutical composition comprising a carboxylic acid or a pharmaceutically acceptable salt thereof as active ingredient, particularly in liquid or solid form, for use in treating nasal congestion, chronic rhinosinusitis, a viral infectious disease of the respiratory tract or an inflammation of the throat, particularly upon administration to a patient in need thereof, particularly wherein said patient is an animal or a human.

The present invention also relates to a carboxylic acid or a pharmaceutically acceptable salt thereof, particularly to a pharmaceutical composition comprising a carboxylic acid or a pharmaceutically acceptable salt thereof as active ingredient, particularly in liquid or solid form, for use in preventing nasal congestion, chronic rhinosinusitis, viral infections of the respiratory tract and/or inflammation of the throat, particularly upon administration to a patient in need thereof, particularly wherein said patient is an animal or a human.

The present invention also relates to a carboxylic acid or a pharmaceutically acceptable salt thereof, particularly to a pharmaceutical composition comprising a carboxylic acid or a pharmaceutically acceptable salt thereof as active ingredient, particularly in liquid or solid form, for use in alleviating the symptoms associated with nasal congestion, chronic rhinosinusitis, viral infections of the respiratory tract and/or inflammation of the throat, particularly upon administration to a patient in need thereof, particularly wherein said patient is an animal or a human.

In a specific embodiment, a decongestive effect is achieved, which is immediate and/or lasting.

In another specific embodiment, a relief from soreness of the throat is achieved, which is immediate and/or lasting.

In various embodiments of the invention, the pharmaceutical composition for use according to the invention and as described herein comprises a pharmaceutically acceptable carrier and/or excipient.

In various further embodiments of the invention, the carboxylic acid for use according to the invention comprises between two and four carbon atoms.

In a specific embodiment, the carboxylic acid for use according to the invention is acetic acid, propionic acid or butyric acid, or a pharmaceutically acceptable salt thereof, preferably propionic acid, or a pharmaceutically acceptable salt thereof.

The pharmaceutical composition for use according to any one of the embodiments disclosed herein may be in liquid form and administered intranasally, otologically, by inhalation or directly to the throat.

In particular, the pharmaceutical composition for use according to any one of the embodiments disclosed herein is applied
(a) intranasally at a dose of about 1000 to 1500 μg per nostril, in particular 1400 μg per nostril per application; and/or
(b) to the throat at a dose of about 1000 to 1500 μg per application, wherein the pharmaceutical composition is administered one to three times per application.

The pharmaceutical composition for use according to any one of the embodiments disclosed herein may be in solid form and administered sublingually or bucally, particularly in form of a lozenge.

In various embodiments, the invention provides a container comprising the carboxylic acid or a pharmaceutically acceptable salt thereof, particularly the pharmaceutical composition according to any one of the embodiments disclosed herein.

In alternative embodiments, the invention provides a method for treating nasal congestion, chronic rhinosinusitis, viral infections of the respiratory tract and/or inflammation of the throat in. a patient, the method comprising administering an effective amount of a carboxylic acid or a pharmaceutically acceptable salt thereof or a pharmaceutical composition comprising a carboxylic acid or a pharmaceutically acceptable salt thereof as disclosed herein in the various embodiments to a patient in need thereof.

In various further embodiments, the invention provides a method for preventing nasal congestion, chronic rhinosinusitis, viral infections of the respiratory tract and/or inflammation of the throat comprising administering an effective amount of a carboxylic acid or a pharmaceutically acceptable salt thereof or a pharmaceutical composition comprising a carboxylic acid or a pharmaceutically acceptable salt thereof as disclosed herein in the various embodiments to a patient in need thereof.

In various further embodiments, the invention provides a method for alleviating the symptoms associated with nasal congestion, chronic rhinosinusitis, viral infections of the respiratory tract and/or inflammation of the throat comprising administering an effective amount of a carboxylic acid or a pharmaceutically acceptable salt thereof or a pharmaceutical composition comprising a carboxylic acid or a pharmaceutically acceptable salt thereof as disclosed herein in the various embodiments to a patient in need thereof.

The carboxylic acid or a pharmaceutically acceptable salt thereof or the pharmaceutical composition may be administered as described herein in any one of the various embodiments.

The carboxylic acid used in the present invention is not particularly limited as long as it is an organic compound that contains a carboxyl group (C(O)OH) and having the general formula of R—C(O)OH, wherein R is a rest attached to the C(O)OH functional group. In particular embodiments of the present invention, R is an alkyl group, optionally having further modifications. In more particular embodiments, R represents a methyl, ethyl or propyl side chain. In a particular embodiment of the present invention, R is an ethyl side chain, the carboxylic acid thus being propionic acid.

Within the meaning of the present invention, the term “alkyl” as such means a straight-chained or branched saturated aliphatic hydrocarbon having from 1 to 10, in particular 1 to 3, carbon atoms, wherein the alkyl group may be unsubstituted or substituted with one or more, same or different, substituents selected from the group consisting of hydroxyl, amino, carboxylic acid, halogen, cyano, or nitro. Preferred are C₁-C₆ alkyl, such as methyl, ethyl, n-propyl, isopropyl, n-butyl, isobutyl, tert-butyl, n-pentyl (amyl), 2-pentyl (sec-pentyl), 3-pentyl, 2-methylbutyl, 3-methylbutyl (=iso-pentyl or iso-amyl), 3-methylbut-2-yl, 2-methylbut-2-yl, 2,2-dimethylpropyl (=neopentyl), n-hexyl, iso-hexyl, sec.-hexyl, tert.-hexyl and the like. Most preferred are C₁-C₃ alkyl, such as methyl, ethyl, n-propyl, isopropyl.

In accordance with the above, in still further specific embodiments, the carboxylic acid according to the invention and as described herein in the various embodiments or aspects is selected from the group consisting of acetic acid, propionic acid, butyric acid, isobutyric acid, 2-hydroxyproirinic acid, dilactic acid, 2-benzyloxypropionic acid, 2-(p-nitrophenyl)-oxy-propionic acid, 3-hydroxypropionic acid, 2,3-dihydroxypropionic acid, methyl 3-hydroxypropionate, ethyl 3-hydroxypropionate, propyl 3-hydroxypropionate, benzyl 3-hydroxypropionate, para-nitrophenyl 3-hydroxypropionate, p-nitrobenzyl 3-hydroxypropionate, polyethylene glycol 3-hydroxypropionate, methyl propionate, ethyl propionate, propyl propionate, benzyl propionate, p-nitrophenyl propionate, p-nitrobenzyl propionate, 2-(4-Isobutylphenyl) propionic acid; or pharmaceutically acceptable salts thereof.

In a particular embodiment of the invention, the compound for use according to the invention and as described herein in the various embodiments or aspects is selected from the group consisting of acetic acid, propionic acid and butyric acid, or pharmaceutically acceptable salts thereof.

The carboxylic acid used in the present invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e. g., carbon-carbon bonds) wherein bond rotation is restricted about that particular linkage, e. g. restriction resulting from the presence of a ring or double bond. Accordingly, all cis-trans and E/Z isomers are expressly included in the present invention. The compounds of this invention may also be represented in multiple tautomeric forms, in such instances, the invention expressly includes all tautomeric forms of the compounds described herein in the various embodiments or aspects, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The carboxylic acid used in the present invention, or the pharmaceutically acceptable salt thereof, or a composition comprising the carboxylic acid according to the invention and as described herein in the various embodiments or aspects, particularly in a therapeutically effective amount, optionally, together with a pharmaceutically acceptable carrier, is used in the treatment and/or prevention of nasal congestion, a viral infection of the respiratory tract and/or inflammation of the throat, and/or in the alleviation of the symptoms associated with nasal congestion, a viral infection of the respiratory tract and/or inflammation of the throat.

Accordingly, in one embodiment, the present invention relates to a carboxylic acid, in particular acetic acid, propionic acid or butyric acid, according to the invention and as described herein in the various embodiments or to a pharmaceutically acceptable salt thereof, or to a composition comprising the carboxylic acid according to the invention and as described herein, or a pharmaceutically acceptable salt thereof, particularly in a therapeutically effective amount, optionally, together with a pharmaceutically acceptable carrier, for use in the treatment and/or prevention of nasal congestion, chronic rhinosinusitis, a viral infection of the respiratory tract and/or inflammation of the throat; and/or in the alleviation of the symptoms associated with nasal congestion, a viral infection of the respiratory tract and/or inflammation of the throat.

The carboxylic acid, in particular acetic acid, propionic acid or butyric acid, according to the invention and as described herein in the various embodiments or to a pharmaceutically acceptable salt thereof, or to a composition comprising the carboxylic acid according to the invention and as described herein, or a pharmaceutically acceptable salt thereof, particularly in a therapeutically effective amount, optionally, together with a pharmaceutically acceptable carrier, is also provided for use in the treatment of cystic fibrosis.

The carboxylic acid, in particular acetic acid, propionic acid or butyric acid, according to the invention and as described herein in the various embodiments or to a pharmaceutically acceptable salt thereof, or to a composition comprising the carboxylic acid according to the invention and as described herein, or a pharmaceutically acceptable salt thereof, particularly in a therapeutically effective amount, optionally, together with a pharmaceutically acceptable carrier, is also provided for use in the alleviation of the symptoms associated of cystic fibrosis.

In this case, it is preferred that the carboxylic acid, in particular acetic acid, propionic acid or butyric acid, according to the invention and as described herein in the various embodiments or the pharmaceutically acceptable salt thereof, or the composition comprising the carboxylic acid according to the invention and as described herein, or the pharmaceutically acceptable salt thereof, particularly in a therapeutically effective amount, optionally, together with a pharmaceutically acceptable carrier, is administered as sinus washes and/or is inhaled as nebulized medication.

Pharmaceutical acceptable carriers are well-known in the art. That is, the person skilled in the art can easily obtain an acceptable carrier for use with the means and methods of the present invention. In a particular embodiment, the pharmaceutical composition of the invention as described herein in the various embodiments or aspects is in form of a solution. Therefore, it is preferred to use pharmaceutical acceptable carriers that are in form of a liquid. Accordingly, the pharmaceutically acceptable carriers include, but are not limited to, water, salt solutions, alcohols, benzyl alcohols, polyethylene glycols. The carrier may also comprise any of the substances described in Remington: The Science and Practice of Pharmacy (Gennaro and Gennaro, Eds, 20th edition, Lippincott Williams & Wilkins, 2000); Theory and Practice of Industrial Pharmacy ((Lachman et al, eds., 3^{rd} edition, Lippincott Williams & Wilkins, 1986); Encyclopedia of Pharmaceutical Technology (Swarbrick and Boylan, eds., 2nd edition, Marcel Dekker, 2002).

Thus, in a first aspect, the present invention provides an aqueous liquid pharmaceutical composition comprising a carboxylic acid according to the invention and as described herein in the various embodiments and a pharmaceutically acceptable carrier.

The pharmaceutical acceptable carrier preferably comprises water. The composition is preferably in the form of a solution. The solution is preferably an aqueous solution.

The composition according to the invention and as described herein in the various embodiments is preferably in the form of a spray, in particular a nasal spray, ear spray or throat spray. In accordance with the present invention, it is possible to make aqueous pharmaceutical compositions which are stable.

The compositions according to the invention and as described herein in the various embodiments are preferably aqueous, which means that the vehicle used is water.

In addition to the carboxylic acid and the pharmaceutical acceptable carrier, the pharmaceutical composition according to the invention and as described herein in the various embodiments may further comprise one or more preservative. It is preferred that the preservative comprises one or more substance selected from the group consisting of benzalkonium chloride, benzethonium chloride, methyl p-hydroxybenzoate, ethyl p-hydroxybenzoate, butyl p-hydroxybenzoate, propyl p-hydroxybenzoate, thimerosal, sodium dehydroacetate and myristyl-gamma-picolinium chloride, sodium benzoate, potassium benzoate, potassium sorbate. Preferably the preservative is benzalkonium chloride. However, it is generally preferred not to use a preservative in the pharmaceutical composition of the invention as described herein in the various embodiments or aspects.

The pharmaceutical composition according to the invention and as described herein in the various embodiments may further comprise one or more buffering agents. The buffering agent may comprise one or more substance selected from the group consisting of sodium hydrogenphosphate, potassium dihydrogenphosphate, dipotassium phosphate, anhydrous sodium dihydrogenphosphate, crystalline sodium dihydrogenphosphate, boric acid, borax, sodium acetate, citric acid, citric anhydride, sodium citrate, sodium glutamate and creatinine. Preferably the buffering agent is citric acid and sodium citrate. The citric acid may be anhydrous. More preferably, the buffering agent is Locke-Ringer solution.

For the purpose of nasal, ear or throat administration a mildly acidic or neutral pH is generally preferred. Preferably the compositions of the present invention have a pH in the range of 4 to 9, more preferably in the range of 6 to 8 and even more preferably in the range of 7.5 to 8.5. Accordingly, the pH of the pharmaceutical composition of the invention as described herein in the various embodiments or aspects may be, for example, 4, 4.5, 5, 5.5, 6, 6.5, 7, 7.5, 8, 8.5 or 9, more preferably 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8.0, 8.1, 8.2, 8.3, 8.4 or 8.5 and even more preferably 7.5, 7.6, 7.7, 7.8, 7.9, 8.0, 8.1, 8.2, 8.3, 8.4 or 8.5.

The compositions according to the invention and as described herein in the various embodiments also possess appropriate isotonicity and viscosity. Preferably compositions according to the present invention have an osmotic pressure of 270 to 550 mOsm/liter. The osmolality of preferable compositions according to the invention is 400 to 550 mosmol/kg. Any suitable isotonic agent and/or thickening agent may be used to achieve appropriate isotonicity and/or viscosity.

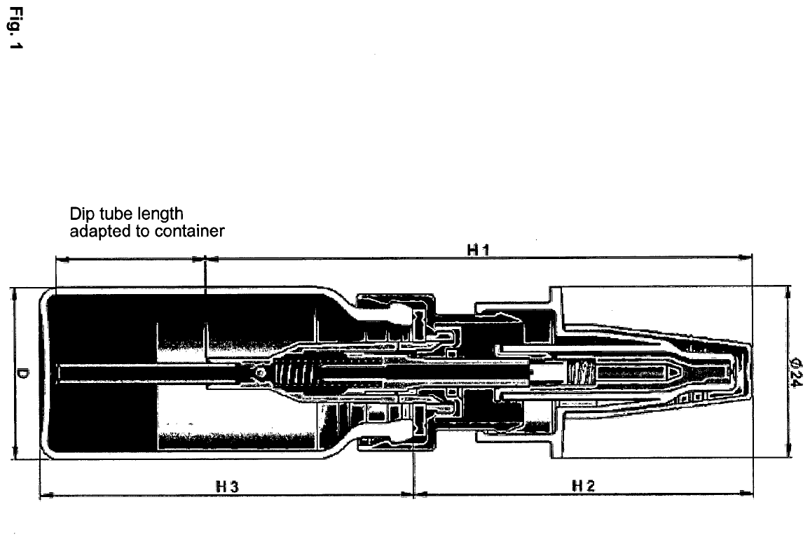

For the purpose of nasal application a composition according to the invention and as described herein in the various embodiments is preferably included in a suitable container. The container is preferably provided with means enabling the application of the contained composition to the nasal mucosa. Suitable applicators are known in the art and include those aiding the administration of liquid nasal compositions in a solution or spray form. For example, a container as shown in FIG. 1 may be used. Since the dosing should be done as accurately as possible, spray form is a more suitable medium. Spray form administrators suitable for use include atomizers, pump-atomizers, aerosols and the like. In a preferred embodiment, one application of the nasal spray extrudes about 140 μl. Thus, the preferred dose per nostril is 140 μl of the pharmaceutical composition. The carboxylic acid as active ingredient may be concentrated at 0.01, 0.02, 0.03, 0.04, 0.05, 0.06, 0.07, 0.08, 0.09, 0.1, 0.11, 0.12, 0.13, 0.14, 0.15, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.89, 0.9, 0.91, 0.92, 0.93, 0.94, 0.95, 0.96, 0.97, 0.98, 0.99, or 1 mg/ml. It may also be concentrated at each 0.01 increment or the specific concentrations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg/ml. Preferably, the concentration is between about 5 mg/ml and about 10 mg/ml.

It will be appreciated, therefore, that the present invention further provides a nasal spray dispenser comprising (i) a housing containing the composition according to the invention and as described herein in the various embodiments and a pharmaceutically acceptable liquid carrier; and (ii) means enabling the application of the composition from within the housing to the nasal mucosa.

For the purpose of throat application a composition according to the invention and as described herein in the various embodiments is preferably included in a suitable container. The container is preferably provided with means enabling the application of the contained composition to the throat. Suitable applicators are known in the art and include those aiding the administration of liquid compositions in a solution or spray form. Since the dosing should be done as accurately as possible, spray form is a more suitable medium. Spray form administrators suitable for use include atomizers, pump-atomizers, aerosols and the like. In a preferred embodiment, one application of the throat spray extrudes about 140 μl. Thus, the preferred dose is 140 μl of the pharmaceutical composition. The carboxylic acid as active ingredient may be concentrated at 0.01, 0.02, 0.03, 0.04, 0.05, 0.06, 0.07, 0.08, 0.09, 0.1, 0.11, 0.12, 0.13, 0.14, 0.15, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.89, 0.9, 0.91, 0.92, 0.93, 0.94, 0.95, 0.96, 0.97, 0.98, 0.99, or 1 mg/ml. It may also be concentrated at each 0.01 increment or the specific concentrations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg/ml. Preferably, the concentration is between about 5 mg/ml and about 10 mg/ml.

For the purpose of in-ear application, i.e. otologic application, a according to the invention and as described herein in the various embodiments is preferably included in a suitable container. The container is preferably provided with means enabling the application of the contained composition to the throat. Suitable applicators are known in the art and include those aiding the administration of liquid compositions in a solution or spray form. Since the dosing should be done as accurately as possible, spray form is a more suitable medium. Spray form administrators suitable for use include atomizers, pump-atomizers, aerosols and the like. In a preferred embodiment, one application of the throat spray extrudes about 140 μl. Thus, the preferred dose is 140 μl of the pharmaceutical composition. The carboxylic acid as active ingredient may be concentrated at 0.01, 0.02, 0.03, 0.04, 0.05, 0.06, 0.07, 0.08, 0.09, 0.1, 0.11, 0.12, 0.13, 0.14, 0.15, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.89, 0.9, 0.91, 0.92, 0.93, 0.94, 0.95, 0.96, 0.97, 0.98, 0.99, or 1 mg/ml. It may also be concentrated at each 0.01 increment or the specific concentrations of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg/ml. Preferably, the concentration is between about 5 mg/ml and about 10 mg/ml. Accordingly, the present invention, in one embodiment, relates to a container suitable for otologic, throat and/or nasal application comprising the pharmaceutical composition of the invention as described herein in the various embodiments or aspects, in particular the pharmaceutical composition comprising as active ingredient a carboxylic acid.

The active ingredients of the present invention can be formulated into the composition as neutralized pharmaceutically acceptable salt forms. Pharmaceutically acceptable salts include, but are not limited to, the acid addition salts, which are formed with inorganic acids such as, for example, hydrochloric, sulfuric or phosphoric acids, or such organic acids as acetic, oxalic, tartaric, mandelic, citric and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Irrespective of the pharmaceutically acceptable carrier used in the present invention or the formulation of the pharmaceutical composition of the invention as described herein in the various embodiments or aspects, it is preferred that the carboxylic acid or the acceptable salt thereof is the only active ingredient comprised in said pharmaceutical composition. The term “active ingredient” within the meaning of the invention, is a pharmaceutical active substance, in particular the carboxylic acid, i.e. a substance that shows a physiological effect when it is absorbed in sufficient amount by the body of an organism.

In this regard, the terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease; (b) inhibiting the disease, i.e. arresting its development; or (c) relieving the disease, i.e. causing regression of the disease.

A “patient” or “subject” for the purposes of the present invention is used interchangeably and meant to include both humans and other animals, particularly mammals, and other organisms. Thus, the methods are applicable to both human therapy and veterinary applications. In the preferred embodiment the patient or subject is a mammal, and in the most preferred embodiment the patient or subject is a human.

The term “propionate” refers to the pharmaceutically acceptable salt of propionic acid such as, for example, the sodium salt of propionic acid.

The term “pharmaceutically acceptable salts” include salts of acidic or basic groups present in compounds of the invention as described herein in the various embodiments or aspects. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

The expressions “pharmaceutical composition” and “therapeutical composition” are used herein interchangeably in the widest sense. They are meant to refer, for the purposes of the present invention, to a therapeutically effective amount of the active ingredient, i.e. the carboxylic acid or a pharmaceutically acceptable salt thereof, optionally, together with a pharmaceutically acceptable carrier or diluent.

It embraces compositions that are suitable for the curative treatment, the control, the amelioration, an improvement of the condition or the prevention of a disease or disorder in a human being or a non-human animal. Thus, it embraces pharmaceutical compositions for the use in the area of human or veterinary medicine. Such a “therapeutic composition” is characterized in that it embraces a carboxylic acid or a physiologically acceptable salt thereof, and optionally a carrier or excipient whereby the salt and the carrier and excipient are tolerated by the target organism that is treated therewith.

The compounds of the present invention and as described herein in the various embodiments and the pharmaceutical compositions containing said compounds may be administered nasally, otologically or to the throat and thus be formulated in a form suitable for such administration routes, as described above.

The pharmaceutical compositions provided herein in the various embodiments may also be administered as controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

The pharmaceutical compositions as described herein in the various embodiments may be used in human and veterinary medicine for treating humans and animals, including avians, non-human primates, dogs, cats, pigs, goats, sheep, cattle, horses, mice, rats and rabbits. It is preferred to treat humans.

Suitable dosages of the pharmaceutical compositions according to the invention and as described herein in the various embodiments will vary depending upon the condition, age and species of the subject, and can be readily determined by those skilled in the art. Such dosage will be adjusted to the individual requirements in each particular case including the specific compound(s) being administered, the route of administration, the condition being treated, as well as the patient being treated. However, the compounds can also be administered as depot preparations (implants, slow-release formulations, etc.) weekly, monthly or at even longer intervals. The appropriate dosage can be determined by conducting conventional model tests, preferably animal models. The daily dosage can be administered as a single dose or in divided doses. It is preferred that a dose of about 140 μl per nostril is applied in case of nasal administration, wherein the concentration of the carboxylic acid as active ingredient is between about 5 mg/ml and 10 mg/ml. The said dose may be applied various times per day, e.g. 1, 2, 3, 4, 5, 6, 7, 8, 9 or 10 times per day depending on the degree of congestion of the nose. Thus, a preferred dosage for intranasal application is a dose of about 1000 to 1500 μg per nostril, in particular 1400 μg per nostril per application. A preferred dosage for application to the throat is a dose of about 1000 to 1500 μg per application, wherein the pharmaceutical composition is administered one to three times per application.

In case of otologic administration or throat application, the dose may be determined using methods well-known in the art.

An effective dose of active ingredient(s) depends at least on the nature of the condition being treated, toxicity, whether the compound(s) is being used prophylactically (lower doses) or against an active condition, the method of delivery, and the pharmaceutical formulation, and will be determined by the clinician using conventional dose escalation studies.

In a particular embodiment of the invention, the dosage is adjusted to achieve an immediate and/or lasting effect, particularly an immediate and/or lasting decongestive effect or an immediate and/or lasting relief from soreness of the throat.

In this regard, “immediate” within the meaning of the present invention refers to an effect occurring without delay or very soon after administration. In particular, the effect occurs 1, 2, 3, 4, 5, 6, 7, 8, 9, 10 or 15 minutes after administration of the pharmaceutical composition of the invention as described herein in the various embodiments or aspects, preferably after 10 minutes or less, more preferably 5 minutes or less. Within the meaning of the present invention, the term “lasting” refers to an effect, in particular a decongestive effect, remaining substantially unchanged over an extended period of time. In particular, the decongestive effect caused by the pharmaceutical composition of the invention as described herein in the various embodiments or aspects lasts over a period of 1, 2, 3, 4, 5, 6, 7, 8, 9 or 10 hours without further administration of the pharmaceutical composition of the invention. As the skilled person will appreciate, a further administration of the pharmaceutical composition of the invention during this period may even extend the period.

The pharmaceutical composition of the invention as described herein in the various embodiments or aspects may have an immediate and lasting effect, in particular where the congestion is mild. In case of hay fever, the effect may be observed only after a more extended period of time, in particular after 20, 25, 30, 35 or 40 minutes after administration of the pharmaceutical composition of the invention.

The effect on viral infections of the respiratory tract and/or inflammation of the throat may also be immediate and/or lasting, as described above. However, the person skilled in the art is well-aware that effects may not be immediately recognized by the patient, depending on the physiological appearance of said effect. That is, a curative effect on viral infections of the respiratory tract and/or inflammation of the throat may be immediate, but physiological appearance may only change after a more extended period of time. Thus, the present invention, in one particular embodiment, provides a pharmaceutical composition having an immediate and/or lasting curative effect on viral infections of the respiratory tract and/or inflammation of the throat, wherein immediate refers to an effect occurring 1, 2, 3, 4, 5, 6, 7, 8, 9, 10 or 15 minutes after administration of the pharmaceutical composition of the invention and lasting refers to an effect over a period of 1, 2, 3, 4, 5, 6, 7, 8, 9 or 10 hours without further administration of the pharmaceutical composition of the invention.

As described above, the effect may be a decongestive effect on the nose. In a further embodiment of the invention, the effect may also be a decline of symptoms such as a dry nose, a runny nose and/or sneezing. Thus, in a further embodiment, the invention relates to a pharmaceutical composition according to the invention and as described herein in the various embodiments having an immediate and/or lasting curative effect on a dry nose, runny nose and/or sneezing, wherein a curative effect on a dry nose refers to a humidification of a dry nose, a curative effect on a runny nose refers to a reduction of such symptoms in particular cessation of a runny nose and a curative effect on sneezing refers to a reduced rate of sneezing, in particular a complete cessation of sneezing.

The pharmaceutical composition of the invention as described herein in the various embodiments or aspects may also be in solid form for inter alia bucal or sublingual administration. When the pharmaceutical composition is used in solid form, the pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, and polyvinyl pyrrolidone. The carrier may also comprise any of the substances described in Remington: The Science and Practice of Pharmacy (Gennaro and Gennaro, Eds, 20th edition, Lippincott Williams & Wilkins, 2000); Theory and Practice of Industrial Pharmacy ((Lachman et al, eds., 3^{rd} edition, Lippincott Williams & Wilkins, 1986); Encyclopedia of Pharmaceutical Technology (Swarbrick and Boylan, eds., 2nd edition, Marcel Dekker, 2002). The fillers can be chosen from, but are not limited to, powdered cellulose, sorbitol, mannitol, various types of lactose, phosphates and the like.

The polymers can be chosen from, but not limited to, hydrophilic or hydrophobic polymers such as derivatives of cellulose (for example methylcellulose, hydroxypropyl cellulose, hypromellose, ethylcellulose); polyvinylpirolidone (for example povidone, crospovidone, copovidone); polymethacrylates (for example Eudragit RS, RL); lypophillic components (for example glyceryl monostearate, glyceryl behenate); and various other substances such as for example hydroxypropyl starch, polyethylene oxide, carrageenan and the like. Most commonly, hydrophilic swelling polymers of suitable viscosity such as hypromellose are used, preferably in amounts above 5%, and more preferably above 8%. Glidants can be chosen from, but not limited to, colloidal silicon dioxide, talc, magnesium stearate, calcium stearate, aluminium stearate, palmitic acid, stearic acid, stearol, cetanol, polyethylene glycol and the like. Lubricants can be chosen from, but not limited to, stearic acid, magnesium stearate, calcium stearate, aluminium stearate, sodium stearyl fumarate, talc, hydrogenated castor oil, polyethylene glycols and the like. A suitable form is a lozenge.

The pharmaceutical composition of the invention as described herein in the various embodiments or aspects is used for treating/preventing nasal congestion, chronic rhinosinusitis, viral infections of the respiratory tract and/or inflammation of the throat.

In this regard, nasal congestion may have various causes. Accordingly, the pharmaceutical composition of the invention as described herein in the various embodiments or aspects may be used for treating/preventing nasal congestion as a symptom of any of these causes. Causes include but are not limited to allergies, like hay fever, allergic reaction to pollen or grass, common cold or influenza, a deviated septum, reaction to medication, Rhinitis medicamentosa, a condition of rebound nasal congestion brought on by extended use of topical decongestants (e.g., oxymetazoline, phenylephrine, xylometazoline, and naphazoline nasal sprays), sinusitis or sinus infection, like in particular a chronic rhinosinusitis, pregnancy as a cause for women to suffer from nasal congestion, nasal polyps, concha bullosa, empty nose syndrome or gastroesophageal reflux disease. A particular embodiment of the invention is the application of the means of the invention to treat/prevent symptoms of hay fever, in particular a congestion, particularly congestion of the nose. That is, the present invention provides a pharmaceutical composition comprising a carboxylic acid or a pharmaceutically acceptable salt thereof as active ingredient for use in treating and/or alleviating and/or preventing nasal congestion caused by hay fever.

Viral infections treated/prevented by the pharmaceutical composition of the invention as described herein in the various embodiments or aspects are particularly those that are taken up by the airway system and manifest themselves in the respiratory tract. Viral infections commonly affect the upper or lower respiratory tract. Although these infections can be classified by the causative virus (eg, influenza), they are generally classified clinically according to syndrome (eg, the common cold, bronchiolitis, croup, nasal congestion, airway congestion). Although specific pathogens commonly cause characteristic clinical manifestations (eg, rhinovirus typically causes the common cold, respiratory syncytial virus (RSV) typically causes bronchiolitis), each can cause many of the viral respiratory syndromes. Accordingly, the pharmaceutical composition can be used for treating/preventing syndroms of bronchiolotis caused by RSV or influenza viruses, parainfluenza viruses, adenoviruses, rhinoviruses; a common cold caused by rhinoviruses, coronaviruses, influenza viruses, parainfluenza viruses, enteroviruses, adenoviruses, human metapneumoviruses; croup caused by parainfluenza viruses, influenza viruses or RSV; influenza-like illness caused by influenza viruses, adenoviruses, parainfluenza viruses; or pneumonia caused by influenza viruses, RSV, adenoviruses, enteroviruses, rhinoviruses, human metapneumoviruses or coronaviruses.

Severity of viral respiratory illness varies widely; severe disease is more likely in the elderly and infants. Morbidity may result directly from viral infection or may be indirect, due to exacerbation of underlying cardiopulmonary conditions or bacterial superinfection of the lung, paranasal sinuses, or middle ear. Accordingly, the pharmaceutical composition of the invention as described herein in the various embodiments or aspects may also be used to prevent any of these diseases/illnesses.

The pharmaceutical composition of the invention as described herein in the various embodiments or aspects may also be used to treat/prevent throat inflammation, in particular pharyngitis. The majority of cases of pharyngitis are due to an infectious organism acquired from close contact with an infected individual. About 40-80% of all cases are infectious cases and can be a feature of many different types of viral infections. Viruses causing pharyngitis comprise, but are not limited to, adenoviruses, the most common of the viral causes. In such cases, typically, the degree of neck lymph node enlargement is modest and the throat often does not appear red, although it is painful. Orthomyxoviridae may also cause pharyngitis. In such cases, a rapid onset of high temperature, headache and generalized ache is diagnosed. A sore throat may be associated. Infectious mononucleosis (“glandular fever”) caused by the Epstein-Barr virus may also be the basis for pharyngitis. This may cause significant lymph gland swelling and an exudative tonsillitis with marked redness and swelling of the throat. The heterophile test, which is known to the person skilled in the art, can be used if this is suspected. The Herpes simplex virus can cause multiple mouth ulcers; measles and common cold caused by rhinovirus, coronavirus, RSV, parainfluenza virus, which all cause infection of the throat, ear, and lungs causing standard cold-like symptoms and often pain. Alternative or additional causes for pharyngitis include bacterial infections of the throat, usually caused by Streptococcus pneumoniae, Haemophilus influenzae, Bordetella pertussis, Bacillus anthracis, Corynebacterium diphtheriae, Neisseria gonorrhoeae, Chlamydophila pneumoniae, and Mycoplasma pneumonia. Pharyngitis may also be caused by non-infectious means such as mechanical, chemical or thermal irritation, for example cold air or acid reflux. Some medications may produce pharyngitis such as pramipexole and antipsychotics. Irrespective of the cause of throat inflammation, in particular pharyngitis, the pharmaceutical composition of the invention as described herein in the various embodiments or aspects may be used to treat/prevent further inflammation or any of the symptoms associated therewith. A further application to the throat is the treatment and/or prevention of congestion due to cystic fibrosis of a patient having cystic fibrosis. In a further embodiment, the throat application may be due to COPD. As is known to the person skilled in the art, cystic fibrosis causes, inter alia, clogging of the airways due to mucus build-up, decreased mucociliary clearance, and resulting inflammation. In the early stages, incessant coughing, copious phlegm production, and decreased ability to exercise are common. In later stages, changes in the architecture of the lung, such as pathology in the major airways (bronchiectasis), further exacerbate difficulties in breathing. Other signs include coughing up blood (hemoptysis), high blood pressure in the lung (pulmonary hypertension), heart failure, difficulties getting enough oxygen to the body (hypoxia), and respiratory failure requiring support with breathing masks, such as bilevel positive airway pressure machines or ventilators. Staphylococcus aureus, Haemophilus influenzae, and Pseudomonas aeruginosa are the three most common organisms causing lung infections in cystic fibrosis. Mucus in the paranasal sinuses is equally thick and may also cause blockage of the sinus passages, leading to infection. This may cause facial pain, fever, nasal congestion, and headaches. Individuals with cystic fibrosis may develop overgrowth of the nasal tissue (nasal polyps) due to inflammation from chronic sinus infections. Recurrent sinonasal polyps can occur in as many as 10% to 25% of cystic fibrosis patients. These polyps can block the nasal passages and increase breathing difficulties. Accordingly, cystic fibrosis may, inter alia, cause symptoms that can be alleviated by the means and methods of the present invention. Therefore, the present invention also relates to a pharmaceutical composition comprising a carboxylic acid for use in treating/preventing/alleviating/reducing symptoms of cystic fibrosis.

Similar symptoms may be observed in patients suffering from chronic obstructive pulmonary disease (COPD), which is a type of obstructive lung disease characterized by long term poor airflow. The main symptoms include shortness of breath and cough with sputum production. The disease is sometimes also referred to as chronic bronchitis. Most cases of COPD can be prevented by reducing exposure to risk factors. This includes decreasing rates of smoking and improving indoor and outdoor air quality. While treatment can slow worsening there is no cure. Current COPD treatments include stopping smoking, vaccinations, respiratory rehabilitation, and often inhaled bronchodilators and steroids. However, there is a lack of an efficient and convenient treatment of symptoms caused by COPD. Thus, the means and methods of the present invention may also be used for treating/preventing/alleviating/reducing symptoms of COPD.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The patent or application file contains at least one drawing executed in color. Copies of this patent or patent application publication with color drawing(s) will be provided by the Office upon request and payment of the necessary fee.

The present invention is also illustrated in some aspects by the following FIGURE.

FIG. 1—Delivery device for intranasal application.
The FIGURE shows the 3K®-System from the company Ursatec Verpackung GmbH (St. Wendel, Germany) in a schematic drawing as exemplary delivery device for the inventive pharmaceutical composition.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention. A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLE 1—SURPRISING DECONGESTIVE EFFECT OF PROPIONATE PER NASAL

In this Example, the decongestive effect of a nasal spray comprising propionate was determined.

The nasal spray prototype has been tested in humans and an immediate improvement of nasal breathing has been noted, which is linked with improved mucus clearance and an apparent ‘opening’ of the upper respiratory tract. This sensation occurs instantaneously indicative of a direct effect of the nasal spray on the epithelial cells, and possibly, mucus. The effect is akin to a nasal decongestion spray. The duration of the affect is 2-6 hours and was clearly more potent and effective than similar nasal spray medical devices already on the market.

These results are following an informal study of 7 independently performed tests in 4 different volunteers.

Given the immediate beneficial effect, it is likely that the mechanism of action is distinct, and in addition to, the enhancement of antiviral immunity via enhanced adaptive immunity previously noted. Without being bound by theory, the mechanism could involve a specific activation of nasal epithelial cells.

### General Study Procedure

Volunteers with an unspecific discomfort in the nose in regards to a congested nose were included in the exploratory study.

The application of the nasal spray for each test occurred as follow: After a slight flexion of the neck, solution was administered into each nostril, via single pressure on the spray pump. Each puff of the nasal spray pump used in this experimental setup relieves about 140 μl. Then the neck was extended and retained in this position for 5-10 seconds. Observation periods were up to 8 hours post administration.

Volunteers documented for each test their observation in regards to tolerability of spray and subjective relief of unspecific discomfort in the nose in case this was applicable. The documented observations were thereafter submitted to the sponsor.

### Results (Descriptive)

### Congested Nose

Volunteer Testing 5 mg/ml Solution

Two independent tests were performed by one volunteer (male) with an unspecific discomfort in the nose in regards to a congested nose. For both independent tests one administration of the 5 mg/ml solution of propionate was performed (140 μl of a 5 mg/ml solution). For both of the tests, following the administration of the 5 mg/ml solution an immediate improvement in breathing could be observed, with a long lasting effect (>2 hr for first test, and up to 6 hrs for second test).

Volunteer Testing 10 mg/ml Solution

Two volunteers (both female) with an unspecific discomfort in the nose in regards to a congested nose tested the 10 mg/ml solution in regards to subjective relief of symptoms. Following the administration of the propionate solution (140 μl of a 10 mg/ml solution), both volunteers independently observed an immediate strong and lasting decongestive effect (>2 hrs).

### Heavily Congested Nose

Volunteer Testing Sequentially all Three Solutions (LR, 5 mg/ml and 10 mg/ml)

One volunteer (female) with an unspecific discomfort in the nose in regards to a heavily congested nose tested all three solutions (LR, 5 mg/ml and 10 mg/ml) in regards to subjective relief of symptoms. The volunteer administered first the LR solution then the 5 mg/ml solution and lastly the 10 mg/ml solution (140 μl of each). Between each administration there was a washout period of 2 hrs.

Following the administration of the LS solution there was next to some moisturizing sensation of the nostril no effect observed. After the administration of the 5 mg/ml solution an immediate minor decongestive effect was observed. And finally following the administration of the 10 mg/ml solution an immediate strong decongestive effect was observed, which lasted for about 2 hrs.

Volunteer Testing Sequentially 5 mg/ml and 10 mg/ml Solution

One volunteer (male) with an unspecific discomfort in the nose in regards to a heavily congested nose tested sequentially the 5 mg/ml and 10 mg/ml solution in regards to subjective relief of symptoms with a washout period of 4 hours between each test.

Following the administration of both solutions (5 mg/ml and 10 mg/ml) an immediate decongestive effect was observed, which lasted for the 5 mg/ml solution about 2.5 hrs and for the 10 mg/ml >2.5 hrs.

Volunteer Testing 10 mg/ml Solution

One volunteer (male) with an unspecific discomfort in the nose in regards to a heavily congested nose tested sequentially the 10 mg/ml solution in regards to subjective relief of symptoms. Following the administration for the first 10 minutes there was no effect observed. However after this first 10 minutes, a clear and strong decongestive effect was observed, which lasted for 2-3 hours.

### Results (Tabular Results, Raw Data)

Test person 1. Female. Age: 18-60

TABLE 1

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 5 | 160219 | Congested | Discomfort | 10 mg/ml | n/a | + | Immediate strong |
| | | Nose | in breathing | | | | and lasting |
| | | | | | | | decongestive |
| | | | | | | | effect (>2 hrs). |

Test person 2. Male. Age: 18-60

TABLE 2

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 9 | 151223 | Heavily | Discomfort | 10 mg/ml | n/a | + | Not immediate, |
| | | Congested | in breathing | | | | yet 10 min after |
| | | Nose | | | | | administration a |
| | | | | | | | clear and strong |
| | | | | | | | decongestive |
| | | | | | | | effect, which |
| | | | | | | | lasted for 2-3 |
| | | | | | | | hours. |

Test person 3. Female. Age: 18-60

TABLE 3

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 10 | 151121 | Heavily | Discomfort | LR | n/a | + | No effect next to |
| | | Congested | in breathing | | | | some moisturizing |
| | | Nose | | | | | sensation of the |
| | | | | | | | nostril. |
| | | | | 5 mg/ml | 2 hrs after | + | Immediate minor |
| | | | | | LR | | decongestive |
| | | | | | | | effect |
| | | | | 10 mg/ml | 2 hrs after | + | Immediate strong |
| | | | | | 5 mg/ml | | decongestive |
| | | | | | | | effect lasting for |
| | | | | | | | about 2 hrs. |
| 11 | 151217 | Congested | Discomfort | 10 mg/ml | n/a | + | Immediate |
| | | Nose | in breathing | | | | decongestive |
| | | | | | | | effect lasting for |
| | | | | | | | about 2 hrs. |

Test person 4. Male. Age: <18

TABLE 4

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 15 | 151121 | Heavily | Discomfort | 5 mg/ml | n/a | + | Immediate |
| | | congested | in breathing | | | | improvement in |
| | | Nose | | | | | breathing, effect |
| | | | | | | | lasted 2.5 hr. |
| | | | | 10 mg/ml | 4 hrs after | + | Immediate |
| | | | | | 5 mg/ml | | improvement in |
| | | | | | | | breathing, with |
| | | | | | | | a long lasting |
| | | | | | | | effect (>2.5 hr) |
| 16 | 151122 | Congested | Discomfort | 5 mg/ml | n/a | + | Immediate |
| | | Nose | in breathing | | | | improvement in |
| | | | | | | | breathing, with |
| | | | | | | | a long lasting |
| | | | | | | | effect (>2 hr) |
| 17 | 160306 | Congested | Discomfort | 5 mg/ml | n/a | + | Immediate |
| | | Nose | in breathing | | | | improvement in |
| | | | | | | | breathing with |
| | | | | | | | a long lasting |
| | | | | | | | effect (up to 6 hr) |

Exploratory Efficacy Trial with Proponent Nasal Spray

### EXAMPLE 2—TEST OF TOLERANCE AND EFFICACY OF THE FINAL MIXTURE OF PRODUCT DEDICATED FOR CLINICAL STUDY

Use of the intended delivery device, which could not be used in mice. Product has no risk—only small cohort of probands for safety reasons.

The same concentrations as in Example 1 are used, i.e. Locke-Ringer/LR with 5 mg/mL Na—P and LR with 10 mg/mL Na—P.

### Test Samples

### Preparation of Solution

After weighing of the appropriate amount of each chemical substance utilizing an analytical balance (Mettler Toledo GmbH, 8606 Greifensee, Switzerland), the chemical substances were added into a glass beaker. Thereafter H2O was added to requested final volume and the solution was mixed utilizing a magnetic stirrer. Immediately after the mixing of the solution, sterile filtration occurred utilizing a 0.22 μm filter (Stericup-GP, 0.22 μm, Polyethersulfon, 150 ml, gamma-sterilized, Catalogue number SCGPU01RE, Merck Millipore Corporation, Merck KGaA, Darmstadt, Germany).

The Locke-Ringer solution had the following composition:

TABLE 5

| | | | | Catalogue | |
|---|---|---|---|---|---|
| Ingredient | mg/ml | | Company | No | Lot No |
| NaCl | 9 | mg | Sigma-Aldrich | S5886-500G | SZBE3170 |
| KCl | 0.42 | mg | Sigma-Aldrich | P5405-500G | SLBH5524V |
| CaCl₂ 2H₂0 | 0.32 | mg | Sigma-Aldrich | C8106-100G | SLBH5904V |
| Dextrose | 2 | mg | Sigma-Aldrich | D9434-250G | SLBH3471V |
| NaHCO₃ | 0.2 | mg | Sigma-Aldrich | S5761-500G | SLBM8267V |

| | |
|---|---|
| Sterile H₂0 | to 1 ml |

The 5 mg/ml Sodium propionate in Locke-Ringer solution had the following composition

TABLE 6

| | | | | Catalogue | |
|---|---|---|---|---|---|
| Ingredient | mg/ml | | Company | No | Lot No |
| NaCl | 9 | mg | Sigma-Aldrich | S5886-500G | SZBE3170 |
| KCl | 0.42 | mg | Sigma-Aldrich | P5405-500G | SLBH5524V |
| CaCl₂ 2H₂0 | 0.32 | mg | Sigma-Aldrich | C8106-100G | SLBH5904V |
| Dextrose | 2 | mg | Sigma-Aldrich | D9434-250G | SLBH3471V |
| NaHCO₃ | 0.2 | mg | Sigma-Aldrich | S5761-500G | SLBM8267V |
| Sodium | 5 | mg | Sigma-Aldrich | P5436-100G | SLBN3602V |
| propionate | | | | | |

| | |
|---|---|
| Sterile H₂0 | to 1 ml |

The 10 mg/ml Sodium propionate in Locke-Ringer solution had the following composition

TABLE 7

| | | | | Catalogue | |
|---|---|---|---|---|---|
| Ingredient | mg/ml | | Company | No | Lot No |
| NaCl | 9 | mg | Sigma-Aldrich | S5886-500G | SZBE3170 |
| KCl | 0.42 | mg | Sigma-Aldrich | P5405-500G | SLBH5524V |
| CaCl₂ 2H₂0 | 0.32 | mg | Sigma-Aldrich | C8106-100G | SLBH5904V |
| Dextrose | 2 | mg | Sigma-Aldrich | D9434-250G | SLBH3471V |
| NaHCO₃ | 0.2 | mg | Sigma-Aldrich | S5761-500G | SLBM8267V |
| Sodium | 10 | mg | Sigma-Aldrich | P5436-100G | SLBN3602V |
| propionate | | | | | |

| | |
|---|---|
| Sterile H₂0 | to 1 ml |

The test item was sodium propionate

TABLE 8

| | |
|---|---|
| Identification: | Sodium propionate |
| | (Sigma-Aldrich, Cat P5436-100G) |
| Test Item Name for Report: | Sodium propionate |
| Description: | Hygroscopic white powder |
| Batch Number: | Lot. SLBN3602V |
| Purity (by Perchlorid Acid | 100% |
| Titration): | |
| Stability of Test Item in | ≥6 months in Locke-Ringer Solution |
| Solution: | |
| Expiry Date (Retest Date): | May 2018 |
| Storage Conditions: | Room temperature |
| Safety Precautions: | Routine hygienic procedures (gloves, |
| | goggles, face mask). |

### Test Item Formulation:

TABLE 9

| | |
|---|---|
| Identification: | Sodium propionate in Locke-Ringer solution |
| Test Item Name for Report: | Proponent Nasal Spray |
| Description: | Clear aqueous colourless liquid |
| Batch Number: | Not applicable - freshly prepared for the |
| | study |
| Purity (HPLC): | 100% |
| Stability of Test Item in | ≥24 months - freshly prepared for the study |
| Solution: | |
| Expiry Date (Retest Date): | Freshly prepared for the study - no re-use |
| Storage Conditions: | In the refrigerator +4° C. |
| Safety Precautions: | Routine hygienic procedures (gloves, |
| | goggles, face mask). |

### Delivery Device Used:

As delivery device the 3K®-System of Ursatec Verpackung GmbH (St. Wendel, Germany) is used.

### Filling of Delivery Device:

20 ml of freshly sterile filtrated Locke-Ringer solution (LR solution, for short “LR”), 5 mg/ml Sodium propionate in Locke-Ringer solution (5 mg/ml solution, for short “5 mg/ml”), and 10 mg/ml Sodium propionate in Locke-Ringer solution (5 mg/ml solution, for short “10 mg/ml”), were added under aseptic conditions into the bottle and leak-proof assembled.

### Characterisation of Participants

### Volunteers Involved in Study:

TABLE 10

| | | | | Study inclusion rational | |
|---|---|---|---|---|---|
| | | | | (safety test or unspecific | |
| Test | | Age | Test | discomfort in the nose of | Solution |
| person | Gender | (years) | No | volunteer) | tested |
| 1 | Female | 18-60 | 1 | Safety test | LR, 5 mg/ml, 10 mg/ml |

| 2 | Sneezing | 5 | mg/ml |
|---|---|---|---|
| 3 | Runny nose | 10 | mg/ml |
| 4 | Runny nose | 10 | mg/ml |
| 5 | Dry nose | LR, 5 mg/ml, 10 mg/ml | |
| 6 | Dry nose | 10 | mg/ml |
| 7 | Congested nose | 10 | mg/ml |

| | | | | | |
|---|---|---|---|---|---|
| 2 | Male | 18-60 | 8 | Safety test | LR, 5 mg/ml, 10 mg/ml |

| | | | |
|---|---|---|---|
| 9 | Heavily congested nose | 10 | mg/ml |

| | | | | | |
|---|---|---|---|---|---|
| 3 | Female | 18-60 | 10 | Heavily congested nose | LR, 5 mg/ml, 10 mg/ml |

| | | | |
|---|---|---|---|
| 11 | Congested nose | 10 | mg/ml |

| | | | | | |
|---|---|---|---|---|---|
| 4 | Male | >60 | 12 | Safety test | LR, 5 mg/ml, 10 mg/ml |
| 5 | Male | 18-60 | 13 | Safety test | 5 mg/ml, 10 mg/ml |
| 6 | Female | 18-60 | 14 | Safety test | 5 mg/ml, 10 mg/ml |
| 7 | Male | <18 | 15 | Heavily congested nose | 5 mg/ml, 10 mg/ml |

| | | | |
|---|---|---|---|
| 16 | Congested nose | 5 | mg/ml |
| 17 | Congested nose | 5 | mg/ml |

### Total Studies Performed:

TABLE 11

| | | | Study | Number of volunteers |
|---|---|---|---|---|
| Study performed | Solution tested | | Repetition | involved |
| Safety test | LR, 5 mg/ml, 10 mg/ml | | 3 | 1 female, 2 males |
| | 5 mg/ml, 10 mg/ml | | 2 | 1 female, 1 male |
| Congested nose | 5 | mg/ml | 2 | 1 male |
| | 10 | mg/ml | 2 | 2 females |
| Heavily congested nose | LR, 5 mg/ml, 10 mg/ml | | 1 | 1 female |
| | 5 mg/ml, 10 mg/ml | | 1 | 1 male |
| | 10 | mg/ml | 1 | 1 male |
| Sneezing | 5 | mg/ml | 1 | 1 female |
| Runny nose | 10 | mg/ml | 2 | 1 female |
| Dry nose | LR, 5 mg/ml, 10 mg/ml | | 1 | 1 female |
| | 10 | mg/ml | 1 | 1 female |

### Case Report Form

Volunteers with an unspecific discomfort in the nose such as enhanced nasal discharge/secretion (runny nose), congested nose, sneezing, itchy nose or feeling of a dry nose were included in the exploratory study. In addition, volunteers without any unspecific discomfort in the nose were included for tolerability testing of the nasal spray.

The application of the nasal spray for each test occurred as follow: After a slight flexion of the neck, solution was administered into each nostril, via single pressure on the spray pump. Then the neck was extended and retained in this position for 5-10 seconds. Observation periods were up to 8 hours post administration.

Volunteers documented for each test their observation in regards to tolerability of spray and subjective relief of unspecific discomfort in the nose in case this was applicable. The documented observations were thereafter submitted to the sponsor.

### Results (Descriptive)

### Drug Safety

Volunteers Testing Sequentially all Three Solutions (LR, 5 mg/ml and 10 mg/ml)

3 different volunteers (two males and one female) without any unspecific discomfort in the nose tested the nasal spray in regards of tolerability. All three solutions (LR, 5 mg/ml and 10 mg/ml) were tested. The volunteers administered first the LR solution then the 5 mg/ml solution and lastly the 10 mg/ml solution. Between each administration there was a washout period of 2 hrs.

Following the administration of LR solution all three volunteers commented that there was no evidence of chemical smell or any salty taste. In addition, all volunteers commented that there was a moisturizing effect on the nostril (alike to any other saline like solution).

Following the administration of the 5 mg/ml solution all volunteers commented that the solution was well tolerated without any unpleasant feeling, smell or salty taste. In addition, all volunteers observed an immediate (slight) decongestive effect, which lasted for 1-2 hrs.

Following the administration of the 10 mg/ml solution all volunteers commented that the solution was well tolerated without any unpleasant feeling, smell or salty taste. In addition, all volunteers observed an immediate strong and lasting decongestive effect observed (>2 hrs).

Volunteers Testing Sequentially 5 mg/ml and 10 mg/ml Solution

2 different volunteers (one male and one female) without any unspecific discomfort in the nose tested sequentially the 5 mg/ml and 10 mg/ml solution in regards of tolerability with a washout period of 4 hours between each test.

Both of the volunteers commented that for both solutions (5 mg/ml and 10 mg/ml) there was (a) no tingling or burning sensations observed within the nasal cavity throughout the period of observation (12 hours), (b) no anosmia was experienced, (c) no headaches was experienced, (d) a very minor characteristic smell of the compound lingered in the nostrils for up to 3 hours post administration was experienced and (e) a slight cooling effect, with a notable sensation of improved inspiratory airflow through the nasal passages was observed. This observed notable sensation of improved inspiratory airflow persisted for the 5 mg/ml solution up to 2 hrs and for the 10 mg/ml solution up to 8 hrs following a single intranasal administration.

### Sneezing

Volunteer Testing 5 mg/ml Solution

One volunteer (female) with an unspecific discomfort in the nose in regards to sneezing tested one administration of the 5 mg/ml solution. Following the administration an immediate ceasing of sneezing was observed with a lasting effect (>2 hrs).

### Runny Nose

Volunteer Testing 10 mg/ml Solution

Two independent tests were performed by one volunteer (female) with an unspecific discomfort in the nose in regards to a runny nose. For both tests one administration of the 10 mg/ml solution was performed. And in both studies, following the administration of the 10 mg/ml solution there was an immediate ceasing of the runny nose observed, which lasted for about 1 hr.

### Dry Nose

Volunteer Testing Sequentially all Three Solution (LS, 5 mg/ml and 10 mg/ml Solution)

One volunteer (female) with an unspecific discomfort in the nose in regards to a dry nose tested all three solutions (LR, 5 mg/ml and 10 mg/ml) in regards to subjective relief of symptoms. The volunteer administered first the LR solution then the 5 mg/ml solution and finally the 10 mg/ml solution. Between each administration there was a washout period of 2 hrs.

Following the administration of the LS solution there was an observation of a short-term moisturizing effect of the nostril (alike to any other saline like solution). After the administration of the 5 mg/ml solution a very minor tingling sensation could be observed, which lasted for about 2 minutes and which was followed by a nice sensation of an enhanced moisturizing, which was sustained for about 1 hr.

Following the administration of the 10 mg/ml solution a very minor tingling sensation could be observed, which lasted for about 3 min and which was followed by a long lasting sensation of enhanced moisturizing, which was sustained for about 2 hr.

Volunteer Testing 10 mg/ml Solution

Same volunteer as above (female) with an unspecific discomfort in the nose in regards to a dry nose examined in a second test the 10 mg/ml solution in regards to subjective relief of symptoms.

Following the administration of the 10 mg/ml solution At first a tingling sensation could be observed, which was followed by a long lasting pleasant sensation of moisturizing. Long lasting effect (>2 hrs).

### Congested Nose

Volunteer Testing 5 mg/ml Solution

Two independent tests were performed by one volunteer (male) with an unspecific discomfort in the nose in regards to a congested nose. For both independent tests one administration of the 5 mg/ml solution was performed. For both of the tests, following the administration of the 5 mg/ml solution an immediate improvement in breathing could be observed, with a long lasting effect (>2 hr for first test, and up to 6 hrs for second test).

Volunteer Testing 10 mg/ml Solution

Two volunteers (both female) with an unspecific discomfort in the nose in regards to a congested nose tested the 10 mg/ml solution in regards to subjective relief of symptoms. Following the administration of the solution, both volunteers independently observed an immediate strong and lasting decongestive effect (>2 hrs).

### Heavily Congested Nose

Volunteer Testing Sequentially all Three Solutions (LR, 5 mg/ml and 10 mg/ml)

One volunteer (female) with an unspecific discomfort in the nose in regards to a heavily congested nose tested all three solutions (LR, 5 mg/ml and 10 mg/ml) in regards to subjective relief of symptoms. The volunteer administered first the LR solution then the 5 mg/ml solution and lastly the 10 mg/ml solution. Between each administration there was a washout period of 2 hrs.

Following the administration of the LS solution there was next to some moisturizing sensation of the nostril no effect observed. After the administration of the 5 mg/ml solution an immediate minor decongestive effect was observed. And finally following the administration of the 10 mg/ml solution an immediate strong decongestive effect was observed, which lasted for about 2 hrs.

Volunteer Testing Sequentially 5 mg/ml and 10 mg/ml Solution

One volunteer (male) with an unspecific discomfort in the nose in regards to a heavily congested nose tested sequentially the 5 mg/ml and 10 mg/ml solution in regards to subjective relief of symptoms with a washout period of 4 hours between each test.

Following the administration of both solutions (5 mg/ml and 10 mg/ml) an immediate decongestive effect was observed, which lasted for the 5 mg/ml solution about 2.5 hrs and for the 10 mg/ml >2.5 hrs.

Volunteer Testing 10 mg/ml Solution

One volunteer (male) with an unspecific discomfort in the nose in regards to a heavily congested nose tested sequentially the 10 mg/ml solution in regards to subjective relief of symptoms. Following the administration for the first 10 minutes there was no effect observed. However after this first 10 minutes, a clear and strong decongestive effect was observed, which lasted for 2-3 hours.

### Results (Tabular Results, Raw Data)

Test Person 1. Female. Age: 18-60

TABLE 12

| | | | Rational | | | | | Description of |
|---|---|---|---|---|---|---|---|---|
| Test | Test | | for study | Solution | | Washout | | subjective relief |
| No | Date | Symptoms | inclusion | tested | | period | Tolerability+/− | of discomfort |
| 1 | 151119 | Safety Test | Tolerability | LR | | n/a | + | No evidence of |
| | | No symptoms | Testing | | | | | chemical smell or |
| | | | | | | | | any salty taste. |
| | | | | | | | | Moisturizing of the |
| | | | | | | | | nostril (alike to |
| | | | | | | | | any other saline |
| | | | | | | | | like solution). |
| | | | | 5 | mg/ml | 2 hrs after | + | No evidence of |
| | | | | | | LR | | chemical smell or |
| | | | | | | | | any salty taste. |
| | | | | | | | | Immediate |
| | | | | | | | | noticeable slight |
| | | | | | | | | decongestive effect, |
| | | | | | | | | which lasted for |
| | | | | | | | | about 1 hr. |
| | | | | 10 | mg/ml | 2 hrs after | + | No evidence of |
| | | | | | | 5 mg/ml | | chemical smell or |
| | | | | | | | | any salty taste. |
| | | | | | | | | Immediate strong |
| | | | | | | | | and lasting |
| | | | | | | | | decongestive |
| | | | | | | | | effect (>2 hrs). |
| 2 | 151120 | Sneezing | Discomfort | 5 | mg/ml | n/a | + | Immediate ceasing |
| | | | due to | | | | | of sneezing. Long |
| | | | sneezing | | | | | lasting effect |
| | | | | | | | | (>2 hrs). |
| 3 | 151121 | Runny | Discomfort | 10 | mg/ml | n/a | + | Immediate ceasing |
| | | Nose | due to | | | | | of runny nose, |
| | | | runny nose | | | | | which lasted for |
| | | | | | | | | about 1 hr. |
| 4 | 160218 | Dry Nose | Discomfort | LR | | n/a | + | Short-term |
| | | | due to dry | | | | | moisturizing of the |
| | | | nose | | | | | nostril (alike to |
| | | | | | | | | any other saline |
| | | | | | | | | like solution). |
| | | | | 5 | mg/ml | 2 hrs after | + | A very minor |
| | | | | | | LR | | tingling sensation, |
| | | | | | | | | which lasts for |
| | | | | | | | | about 2 min and |
| | | | | | | | | which is followed |
| | | | | | | | | by a nice sensation |
| | | | | | | | | of an enhanced |
| | | | | | | | | moisturizing, |
| | | | | | | | | which is s |
| | | | | | | | | sustained for |
| | | | | | | | | about 1 hr. |
| | | | | 10 | mg/ml | 2 hrs after | + | At first a tingling |
| | | | | | | 5 mg/ml | | sensation, which |
| | | | | | | | | lasts for about 3 |
| | | | | | | | | min and which is |
| | | | | | | | | followed by a long |
| | | | | | | | | lasting sensation |
| | | | | | | | | of beneficial |
| | | | | | | | | moisturizing. Long |
| | | | | | | | | lasting effect |
| | | | | | | | | (>2 hrs). |
| 5 | 160219 | Congested | Discomfort | 10 | mg/ml | n/a | + | Immediate strong |
| | | Nose | in breathing | | | | | and lasting |
| | | | | | | | | decongestive |
| | | | | | | | | effect (>2 hrs). |
| 6 | 160220 | Runny | Discomfort | 10 | mg/ml | n/a | + | Immediate ceasing |
| | | Nose | due to | | | | | of runny nose, |
| | | | runny nose | | | | | which lasted for |
| | | | | | | | | about 30 min. |
| 7 | 160308 | Dry Nose | Discomfort | 10 | mg/ml | n/a | + | At first a tingling |
| | | | due to dry | | | | | sensation, which |
| | | | nose | | | | | follows by a long |
| | | | | | | | | lasting sensation |
| | | | | | | | | of moisturizing. |
| | | | | | | | | Long lasting effect |
| | | | | | | | | (>2 hrs). |

Test Person 2. Male. Age: 18-60

TABLE 13

| | | | Rational | | | | | Description of |
|---|---|---|---|---|---|---|---|---|
| Test | Test | | for study | Solution | | Washout | | subjective relief |
| No | Date | Symptoms | inclusion | tested | | period | Tolerability+/− | of discomfort |
| 8 | 151120 | Safety Test | Tolerability | LR | | n/a | + | No evidence of |
| | | No symptoms | Testing | | | | | chemical smell or |
| | | | | | | | | any salty taste. |
| | | | | | | | | Moisturizing of the |
| | | | | | | | | nostril (alike to any |
| | | | | | | | | other saline like |
| | | | | | | | | solution). |
| | | | | 5 | mg/ml | 2 hrs after | + | No evidence of |
| | | | | | | LR | | chemical smell or |
| | | | | | | | | any salty taste. |
| | | | | | | | | Immediate |
| | | | | | | | | noticeable slight |
| | | | | | | | | decongestive |
| | | | | | | | | effect, which lasted |
| | | | | | | | | for about 1 hr. |
| | | | | 10 | mg/ml | 2 hrs after | + | No evidence of |
| | | | | | | 5 mg/ml | | chemical smell nor |
| | | | | | | | | any salty taste. |
| | | | | | | | | Immediate strong |
| | | | | | | | | and lasting |
| | | | | | | | | decongestive effect |
| | | | | | | | | (>2 hrs). |
| 9 | 151223 | Heavily | Discomfort | 10 | mg/ml | n/a | + | Not immediate, yet |
| | | Congested | in breathing | | | | | 10 min after |
| | | Nose | | | | | | administration a |
| | | | | | | | | clear and strong |
| | | | | | | | | decongestive effect, |
| | | | | | | | | which lasted |
| | | | | | | | | for 2-3 hours. |

Test Person 3. Female. Age: 18-60

TABLE 14

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 10 | 151121 | Heavily | Discomfort | LR | n/a | + | No effect next to |
| | | Congested | in breathing | | | | some moisturizing |
| | | Nose | | | | | sensation of the |
| | | | | | | | nostril. |
| | | | | 5 mg/ml | 2 hrs after | + | Immediate minor |
| | | | | | LR | | decongestive effect |
| | | | | 10 mg/ml | 2 hrs after | + | Immediate strong |
| | | | | | 5 mg/ml | | decongestive effect |
| | | | | | | | lasting for about 2 |
| | | | | | | | hrs. |
| 11 | 151217 | Congested | Discomfort | 10 mg/ml | n/a | + | Immediate |
| | | Nose | in breathing | | | | decongestive effect |
| | | | | | | | lasting for about 2 |
| | | | | | | | hrs. |

Test Person 4. Male. Age: >60

TABLE 15

| | | | Rational | | | | Description of |
|---|---|---|---|---|---|---|---|
| Test | Test | | for study | Solution | Washout | Tolerability | subjective relief |
| No | Date | Symptoms | inclusion | tested | period | +/− | of discomfort |
| 12 | 151124 | Safety test | Tolerability | LR | n/a | + | No evidence of |
| | | | Testing | | | | smell or salty |
| | | | | | | | taste. A wetting |
| | | | | | | | effect. |
| | | | | 5 mg/ml | 2 hrs after | + | Well tolerated |
| | | | | | LR | | administration, |
| | | | | | | | no unpleasant |
| | | | | | | | feeling, no smell, |
| | | | | | | | no salty taste. |
| | | | | | | | Immediate |
| | | | | | | | decongestive |
| | | | | | | | effect lasting for |
| | | | | | | | 1-2 hrs. |
| | | | | 10 mg/ml | 4 hrs after | + | The clearance of |
| | | | | | 5 mg/ml | | the nose is quick |
| | | | | | | | and stronger and |
| | | | | | | | the effect lasts |
| | | | | | | | at least two |
| | | | | | | | hours! At the |
| | | | | | | | beginning of the |
| | | | | | | | administration |
| | | | | | | | there is a slight |
| | | | | | | | salty taste but it |
| | | | | | | | is transient, not |
| | | | | | | | unpleasant or |
| | | | | | | | uncomfortable. |
| | | | | | | | There is no smell. |

Test Person 5. Male. Age: 18-60

TABLE 16

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 13 | 151216 | Safety test | Tolerability | 5 mg/ml | n/a | + | (a) No tingling or |
| | | | Testing | | | | burning sensations |
| | | | | | | | within the nasal |
| | | | | | | | cavity throughout |
| | | | | | | | the period of |
| | | | | | | | observation. |
| | | | | | | | (b) No anosmia was |
| | | | | | | | experienced. |
| | | | | | | | (c) No headaches |
| | | | | | | | was experienced. |
| | | | | | | | (d) A very minor |
| | | | | | | | characteristic smell |
| | | | | | | | of the compound |
| | | | | | | | lingered in the |
| | | | | | | | nostrils for up to 3 |
| | | | | | | | hours post |
| | | | | | | | administration. |
| | | | | | | | (e) A Slight cooling |
| | | | | | | | effect, with a notable |
| | | | | | | | sensation of |
| | | | | | | | improved inspiratory |
| | | | | | | | airflow through the |
| | | | | | | | nasal passages |
| | | | | | | | were observed. |
| | | | | | | | These persisted for |
| | | | | | | | up to 2 hours after |
| | | | | | | | the 5 mg/ml dose. |
| | | | | 10 mg/ml | 4 hrs after | + | (a) No tingling or |
| | | | | | 5 mg/ml | | burning sensations |
| | | | | | | | within the nasal |
| | | | | | | | cavity throughout |
| | | | | | | | the period of |
| | | | | | | | observation. |
| | | | | | | | (b) No anosmia was |
| | | | | | | | experienced. |
| | | | | | | | (c) No headaches |
| | | | | | | | was experienced. |
| | | | | | | | (d) A very minor |
| | | | | | | | characteristic smell |
| | | | | | | | of the compound |
| | | | | | | | lingered in the |
| | | | | | | | nostrils for up to 3 |
| | | | | | | | hours post |
| | | | | | | | administration. |
| | | | | | | | (e) A Slight cooling |
| | | | | | | | effect, with a notable |
| | | | | | | | sensation of |
| | | | | | | | improved inspiratory |
| | | | | | | | airflow through the |
| | | | | | | | nasal passages |
| | | | | | | | were observed. |
| | | | | | | | These persisted for |
| | | | | | | | up to 8 hours after a |
| | | | | | | | single intranasal |
| | | | | | | | administration of the |
| | | | | | | | 10 mg/ml dose. |

Test Person 6. Female. Age: 18-60

TABLE 17

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 13 | 151216 | Safety test | Tolerability | 5 mg/ml | n/a | + | (a) No tingling or |
| | | | Testing | | | | burning sensations |
| | | | | | | | within the nasal |
| | | | | | | | cavity throughout |
| | | | | | | | the period of |
| | | | | | | | observation. |
| | | | | | | | (b) No anosmia was |
| | | | | | | | experienced. |
| | | | | | | | (c) No headaches |
| | | | | | | | was experienced. |
| | | | | | | | (d) A very minor |
| | | | | | | | characteristic smell |
| | | | | | | | of the compound |
| | | | | | | | lingered in the |
| | | | | | | | nostrils for up to 3 |
| | | | | | | | hours post |
| | | | | | | | administration. |
| | | | | | | | (e) A Slight cooling |
| | | | | | | | effect, with a notable |
| | | | | | | | sensation of |
| | | | | | | | improved inspiratory |
| | | | | | | | airflow through the |
| | | | | | | | nasal passages |
| | | | | | | | were observed. |
| | | | | | | | These persisted for |
| | | | | | | | up to 2 hours after |
| | | | | | | | the 5 mg/ml dose. |
| | | | | 10 mg/ml | 4 hrs after | + | (a) No tingling or |
| | | | | | 5 mg/ml | | burning sensations |
| | | | | | | | within the nasal |
| | | | | | | | cavity throughout |
| | | | | | | | the period of |
| | | | | | | | observation. |
| | | | | | | | (b) No anosmia was |
| | | | | | | | experienced. |
| | | | | | | | (c) No headaches |
| | | | | | | | was experienced. |
| | | | | | | | (d) A very minor |
| | | | | | | | characteristic smell |
| | | | | | | | of the compound |
| | | | | | | | lingered in the |
| | | | | | | | nostrils for up to 3 |
| | | | | | | | hours post |
| | | | | | | | administration. |
| | | | | | | | (e) A Slight cooling |
| | | | | | | | effect, with a notable |
| | | | | | | | sensation of |
| | | | | | | | improved inspiratory |
| | | | | | | | airflow through the |
| | | | | | | | nasal passages |
| | | | | | | | were observed. |
| | | | | | | | These persisted for |
| | | | | | | | up to 8 hours after a |
| | | | | | | | single intranasal |
| | | | | | | | administration of the |
| | | | | | | | 10 mg/ml dose. |

Test Person 7. Male. Age: <18

TABLE 18

| | | | Rational | | | | | Description of |
|---|---|---|---|---|---|---|---|---|
| Test | Test | | for study | Solution | | Washout | | subjective relief |
| No | Date | Symptoms | inclusion | tested | | period | Tolerability+/− | of discomfort |
| 15 | 151121 | Heavily | Discomfort | 5 | mg/ml | n/a | + | Immediate |
| | | congested | in breathing | | | | | improvement in |
| | | Nose | | | | | | breathing, effect |
| | | | | | | | | lasted 2.5 hr. |
| | | | | 10 | mg/ml | 4 hrs after | + | Immediate |
| | | | | | | 5 mg/ml | | improvement in |
| | | | | | | | | breathing, with a |
| | | | | | | | | long lasting effect |
| | | | | | | | | (>2.5 hr) |
| 16 | 151122 | Congested | Discomfort | 5 | mg/ml | n/a | + | Immediate |
| | | Nose | in breathing | | | | | improvement in |
| | | | | | | | | breathing, with a |
| | | | | | | | | long lasting effect |
| | | | | | | | | (>2 hr) |
| 17 | 160306 | Congested | Discomfort | 5 | mg/ml | n/a | + | Immediate |
| | | Nose | in breathing | | | | | improvement in |
| | | | | | | | | breathing with a |
| | | | | | | | | long lasting effect |
| | | | | | | | | (up to 6 hr) |

### Conclusions

Using the product with 10 mg/mL this clearance effect is perceived as more effective and longer lasting. The slight salty taste which is not unpleasant may generate the impression and the subjective feeling at the users that this presentation of the product is more “strong” but in an advantageous sense.

No aromatization seems to be necessary. Thus, the product is a pure natural product without any addition of preservatives and/or flavors.

### EXAMPLE 3—CARBOXYLIC ACID APPLIED BY A THROAT SPRAY

Volunteers were applied solutions as prepared above as a spray directly applied to the throat. A 10 mg/ml Na—P solution was applied to volunteers with an unspecific discomfort such as sore throat.

### Volunteers Involved in Study:

TABLE 19

| | | | | Study inclusion rational | |
|---|---|---|---|---|---|
| | | | | (safety test or unspecific | |
| Test | | Age | Test | discomfort in the nose of | Solution |
| person | Gender | (years) | No | volunteer) | tested |
| 1 | Female | 18-60 | 1 | Sore throat | 10 mg/ml |
| | | | 2 | Sore throat | 10 mg/ml |
| | | | 3 | Sore throat | 10 mg/ml |
| 2 | Male | 18-60 | 4 | Sore throat | 10 mg/ml |
| 3 | Female | 18-60 | 5 | Sore throat | 10 mg/ml |
| | | | 6 | Sore throat | 10 mg/ml |

### Total Studies Performed:

TABLE 20

| | | Study | Number of volunteers |
|---|---|---|---|
| Study performed | Solution tested | Repetition | involved |
| Sore throat | 10 mg/ml | 3 | 1 female |
| | 10 mg/ml | 1 | 1 male |
| | 10 mg/ml | 2 | 1 female |

### General Study Procedure:

Volunteers with an unspecific discomfort in the throat such as sore throat were included in the exploratory study.

The application of the throat spray for each test occurred as follow: After a slight flexion of the neck, solution was administered into throat, via single pressure on the spray pump. Then the neck was extended and retained in this position for 5-10 seconds. Observation periods were up to 8 hours post administration.

Volunteer documented for each test their observation in regards to tolerability of spray and subjective relief of unspecific discomfort in the ear in case this was applicable. The documented observations were thereafter submitted to the sponsor.

### Results

Volunteer No 1 Testing 10 mg/ml Solution

Three independent tests were performed by one volunteer (female) with an unspecific discomfort in the throat such as sore throat. For all three independent tests three administration of the 10 mg/ml solution were performed. And for all three tests, following the administration of the 10 mg/ml solution an immediate relief from soreness could be observed, which lasted for 15-60 min.

Volunteer No 2 Testing 10 mg/ml Solution

One test was performed by one volunteer (male) with an unspecific discomfort in the throat such as sore throat. The volunteer administered one pump of the 10 mg/ml solution. Following the administration of the 10 mg/ml solution an immediate relief from soreness could be observed, which lasted for 15-60 min.

Volunteer No 2 Testing 10 mg/ml Solution

Two independent tests were performed by one volunteer (female) with an unspecific discomfort in the throat such as sore throat. For both independent tests volunteer administered one pump of the 10 mg/ml solution. For both of the tests, following the administration of the 10 mg/ml solution an immediate relief from soreness could be observed, which lasted for 15-60 min.

Test Person 1. Female. Age: 18-60

TABLE 21

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 1 | 151121 | Sore | Discomfort | 10 mg/ml | n/a | + | Immediate relief from |
| | | throat | due to sore | | | | soreness, which lasted |
| | | | throat | | | | for 15-60 min. |
| 2 | 160219 | Sore | Discomfort | 10 mg/ml | n/a | + | Immediate relief from |
| | | throat | due to sore | | | | soreness, which lasted |
| | | | throat | | | | for 15-60 min. |
| 3 | 160220 | Sore | Discomfort | 10 mg/ml | n/a | + | Immediate relief from |
| | | throat | due to sore | | | | soreness, which lasted |
| | | | throat | | | | for 15-60 min. |

Test Person 2. Male. Age: 18-60

TABLE 22

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 4 | 151223 | Sore | Discomfort | 10 mg/ml | n/a | + | Immediate relief from |
| | | throat | due to sore | | | | soreness, which lasted |
| | | | throat | | | | for 15-60 min. |

Test Person 3. Female. Age: 18-60

TABLE 23

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 5 | 151121 | Sore throat | Discomfort | 10 mg/ml | n/a | + | Immediate relief from |
| | | | due to sore | | | | soreness, which lasted |
| | | | throat | | | | for 15-60 min. |
| 6 | 151217 | Sore throat | Discomfort | 10 mg/ml | n/a | + | Immediate relief from |
| | | | due to sore | | | | soreness, which lasted |
| | | | throat | | | | for 15-60 min. |

### EXAMPLE 4—CARBOXYLIC ACID APPLIED TO TREAT CONGESTED EAR CAUSED BY HEY FEVER

Use of 10 mg/ml Na—P solution by volunteers with an unspecific discomfort such as congested ear (blocked ear) caused by allergic coryza (hay fever). Test samples were prepared as described above.

### Volunteers Involved in Study

TABLE 24

| | | | | Study inclusion rational | |
|---|---|---|---|---|---|
| | | | | (safety test or unspecific | |
| Test | | Age | Test | discomfort in the nose of | Solution |
| person | Gender | (years) | No | volunteer) | tested |
| 1 | Female | 18-60 | 1 | Congested ear | 10 mg/ml |
| | | | | caused by allergic coryza | |
| | | | | (hey fever) | |
| | | | 2 | Congested ear | 10 mg/ml |
| | | | | caused by allergic coryza | |
| | | | | (hey fever) | |

### Total Studies Performed:

TABLE 25

| | | Study | Number of volunteers |
|---|---|---|---|
| Study performed | Solution tested | Repetition | involved |
| Congested ear | 10 mg/ml | 2 | 1 female |
| caused by allergic | | | |
| coryza (hey fever) | | | |

### General Study Procedure:

Volunteers with an unspecific discomfort in the ear such as congested or blocked ear caused by allergic coryza (hey fever) were included in the exploratory study.

The application of the otological spray for each test occurred as follow: After positioning head straight and upright and the neck straight, solution was administered into each ear cavity, via single pressure on the spray pump. Then the upright position was maintained for 5-10 seconds. Observation periods were up to 8 hours post administration.

Volunteer documented for each test their observation in regards to tolerability of spray and subjective relief of unspecific discomfort in the ear in case this was applicable. The documented observations were thereafter submitted to the sponsor.

### Results

### Congested Ear Caused by Allergic Coryza (Hey Fever)

Volunteer Testing 10 mg/ml Solution (One Female, Two Independent Tests)

Two independent tests were performed by one volunteer (female) with an unspecific discomfort in the ear in regards to congested ear caused by allergic coryza (hey fever). For both tests one administration of the 10 mg/ml solution was performed into each ear cavity. For both of the tests, following the administration of the 10 mg/ml solution an immediate decongestive effect of ear could be observed, with a lasting effect (>2 hr).

Test Person 1. Female. Age: 18-60

TABLE 26

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 1 | 160325 | Congested | Congested | 10 mg/ml | n/a | + | Well tolerated |
| | | Ear | ear caused | | | | administration, no |
| | | | by allergic | | | | unpleasant feeling. |
| | | | coryza | | | | Immediate |
| | | | (hey fever) | | | | decongestive effect |
| | | | | | | | lasting for >2 hrs. |
| 2 | 160326 | Congested | Congested | 10 mg/ml | n/a | + | Well tolerated |
| | | Ear | ear caused | | | | administration, no |
| | | | by allergic | | | | unpleasant feeling. |
| | | | coryza | | | | Immediate |
| | | | (hey fever) | | | | decongestive effect |
| | | | | | | | lasting for >2 hrs. |

### EXAMPLE 5—TREATMENT OF CONGESTED NOSE CAUSED BY HAY FEVER USING A NASAL SPRAY

Use of 10 mg/ml Na—P solution by volunteers with an unspecific discomfort such as congested nose caused by allergic coryza (hay fever). Test samples were prepared as described above.

### Volunteers Involved in Study:

TABLE 27

| | | | | Study inclusion rational | |
|---|---|---|---|---|---|
| | | | | (safety test or unspecific | |
| Test | | Age | Test | discomfort in the nose of | Solution |
| person | Gender | (years) | No | volunteer) | tested |
| 1 | Female | 18-60 | 1 | Congested nose | 10 mg/ml |
| | | | | caused by allergic coryza | |
| | | | | (hey fever) | |
| | | | 2 | Congested nose | 10 mg/ml |
| | | | | caused by allergic coryza | |
| | | | | (hey fever) | |
| 2 | Male | 18-60 | 3 | Congested nose | 10 mg/ml |
| | | | | caused by allergic coryza | |
| | | | | (hey fever) | |

### Total Studies Performed:

TABLE 28

| | | Study | Number of volunteers |
|---|---|---|---|
| Study performed | Solution tested | Repetition | involved |
| Congested nose | 10 mg/ml | 2 | 1 female |
| caused by allergic | 10 mg/ml | 1 | 1 male |
| coryza (hey fever) | | | |

### General Study Procedure:

Volunteers with an unspecific discomfort in the nose such as congested or stuffy nose caused by allergic coryza (hey fever) were included in the exploratory study.

The application of the nasal spray for each test occurred as follow: After a slight flexion of the neck, solution was administered into each nostril, via single pressure on the spray pump. Then the neck was extended and retained in this position for 5-10 seconds. Observation periods were up to 8 hours post administration.

Volunteers documented for each test their observation in regards to tolerability of spray and subjective relief of unspecific discomfort in the nose in case this was applicable. The documented observations were thereafter submitted to the sponsor.

### Results

### Congested Nose Caused by Allergic Coryza (Hey Fever)

Volunteer Testing 10 mg/ml Solution (One Female, Two Independent Tests)

Two independent tests were performed by one volunteer (female) with an unspecific discomfort in the nose in regards to congested nose caused by allergic coryza (hey fever). For both tests one administration of the 10 mg/ml solution was performed. In both studies, following the administration of the 10 mg/ml solution there was a decongestive effect of the nose observed, which started around 30 min following the administration of the 10 mg/ml solution and which had a long-lasting effect (6-8 hr).

Volunteer Testing 10 mg/ml Solution (One Male, One Independent Tests)

One test was performed by one volunteer (male) with a discomfort in the nose in regards to congested nose caused by allergic coryza (hey fever). One administration of the 10 mg/ml solution was performed. Similarly as to the observation described by other volunteer, following the administration of the 10 mg/ml solution there was a decongestive effect of the nose observed, which started around 30 min following the administration of the 10 mg/ml solution and which had a long-lasting effect (6-8 hr).

Test Person 1. Female. Age: 18-60

TABLE 29

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 1 | 160325 | Congested | Congested | 10 mg/ml | n/a | + | Not immediate, yet |
| | | Nose | nose caused | | | | 30 min after |
| | | | by allergic | | | | administration a |
| | | | coryza | | | | clear and strong |
| | | | (hey fever) | | | | decongestive effect, |
| | | | | | | | with a long lasting |
| | | | | | | | effect (6-8 hours). |
| 2 | 160326 | Congested | Congested | 10 mg/ml | n/a | + | Not immediate, yet |
| | | Nose | nose caused | | | | 30 min after |
| | | | by allergic | | | | administration a |
| | | | coryza | | | | clear and strong |
| | | | (hey fever) | | | | decongestive effect, |
| | | | | | | | with a long lasting |
| | | | | | | | effect (6-8 hours). |

Test Person 2. Male. Age: 18-60

TABLE 30

| | | | Rational | | | | Description of |
|---|---|---|---|---|---|---|---|
| Test | Test | | for study | Solution | Washout | | subjective relief |
| No | Date | Symptoms | inclusion | tested | period | Tolerability+/− | of discomfort |
| 3 | 360410 | Congested | Congested | 10 mg/ml | n/a | + | Not immediate, |
| | | Nose | nose caused | | | | yet 30 min after |
| | | | by allergic | | | | administration a |
| | | | coryza (hey | | | | clear and strong |
| | | | fever) | | | | decongestive effect, |
| | | | | | | | with a long lasting |
| | | | | | | | effect (6-8 hours). |

### EXAMPLE 6—ALLEVIATING SYMPTOMS OF CHRONIC RHINOSINUSITIS

Use of 10 mg/ml Na—P solution by volunteers suffering from chronic rhinosinusitis and a strong unspecific nasal discomfort caused thereby.

The volunteers were given a one time administration of 140 μl of the 10 mg/ml solution in both nostrils. The volunteers experienced a strong decongestive effect within 5-30 minutes following the application. The decongestive effect lasted for more than 60 minutes.

1-14. (canceled)

15. A method for treating nasal congestion, viral infections of the respiratory tract, inflammation of the throat in a patient, or a combination thereof, the method comprising administering to a patient in need thereof an effective amount of a pharmaceutical composition comprising a carboxylic acid or a pharmaceutically acceptable salt thereof.

16-18. (canceled)

19. The method of claim 15, wherein the carboxylic acid or a pharmaceutically acceptable salt thereof comprises between two and four carbon atoms.

20. The method of claim 15, wherein the carboxylic acid or pharmaceutically acceptable salt thereof is one or more of acetic acid, propionic acid, butyric acid, isobutyric acid, 2-hydroxyproirinic acid, dilactic acid, 2-benzyloxypropionic acid, 2-(p-nitrophenyl)-oxy-propionic acid, 3-hydroxypropionic acid, 2,3-dihydroxypropionic acid, methyl 3-hydroxypropionate, ethyl 3-hydroxypropionate, propyl 3-hydroxypropionate, benzyl 3-hydroxypropionate, para-nitrophenyl 3-hydroxypropionate, p-nitrobenzyl 3-hydroxypropionate, polyethylene glycol 3-hydroxypropionate, methyl propionate, ethyl propionate, propyl propionate, benzyl propionate, p-nitrophenyl propionate, p-nitrobenzyl propionate, 2-(4-Isobutylphenyl) propionic acid, or pharmaceutically acceptable salt of any thereof.

21. The method of claim 15, wherein the carboxylic acid or pharmaceutically acceptable salt thereof is one or more of acetic acid, propionic acid, butyric acid, or a pharmaceutically acceptable salt of any thereof.

22. The method of claim 15, wherein the carboxylic acid is propionic acid or a pharmaceutically acceptable salt thereof.

23. The method of claim 15, wherein the pharmaceutical composition is in liquid form or solid form.

24. The method of claim 15, wherein the pharmaceutical composition is in the form of a spray.

25. The method of claim 15, wherein the pharmaceutical composition is in the form of an aqueous solution.

26. The method of claim 15, wherein the pharmaceutical composition is in the form of a lozenge.

27. The method of claim 15, wherein the pharmaceutical composition further comprises a pharmaceutically acceptable carrier.

28. The method of claim 15, wherein the pharmaceutical composition further comprises a preservative.

29. The method of claim 15, wherein the pharmaceutical composition has a pH in the range of 6 to 8.

30. The method of claim 15, wherein the pharmaceutical composition has an osmotic pressure of 270 to 550 mOsm/liter.

31. The method of claim 15, wherein the pharmaceutical composition is in liquid form and is administered to the patient intranasally, ontologically, by inhalation, or directly to the patient's throat.

32. The method of claim 15, wherein the pharmaceutical composition is in liquid form and is administered to the patient intranasally at a dose of about 1000 to 1500 μg per nostril, in particular 1400 μg per nostril per application.

33. The method of claim 15, wherein the pharmaceutical composition is in liquid form and is administered to the patient's throat at a dose of about 1000 to 1500 μg per application, wherein the pharmaceutical composition is administered one to three times per application.

34. The method of claim 15, wherein the pharmaceutical composition is in solid form and is administered to the patient sublingually or bucally.

35. The method of claim 15, wherein a decongestive effect is achieved in the patient, wherein the decongestive effect occurs within 1-15 minutes, lasts at least 2-6 hours, or both.

36. The method of claim 15, wherein inflammation of the throat is decreased thereby providing a relief from soreness for the patient.

37. The method of claim 15, wherein the patient has chronic rhinosinusitis.
